(19) United States
(12) Patent Application Publication
(10) Pub. No.: US 2023/0381371 A1
(43) Pub. Date: Nov. 30, 2023

(54) POLYMER HYDROGEL WITH SLOW-RELEASE FUNCTION AND PREPARATION METHOD AND USE THEREOF

(71) Applicant: SHANGHAI CHUANGSHI MEDICAL TECHNOLOGY (GROUP) CO, LTD, Shanghai (CN)

(72) Inventors: Qian CHEN, Shanghai (CN); Litao FAN, Shanghai (CN); Chengyu ZHANG, Shanghai (CN); Yong YOU, Shanghai (CN); Yajian WU, Shanghai (CN)

(73) Assignee: SHANGHAI CHUANGSHI MEDICAL TECHNOLOGY (GROUP) CO, LTD, Shanghai (CN)

(21) Appl. No.: 18/033,371

(22) PCT Filed: Mar. 11, 2022
(86) PCT No.: PCT/CN2022/080351

§ 371 Date: Apr. 24, 2023

(30) Foreign Application Priority Data
Mar. 12, 2021 (CN) 202110269382.8
Mar. 12, 2021 (CN) 202110271604.X

(51) Int. Cl. A61L 26/00

(52) U.S. Cl. CPC A61L 26/008; A61L 26/0066; A61L 26/0052; A61L 2300/204; A61L 2300/216; A61L 2300/602; A61L 2300/802

(57) ABSTRACT

A polymer hydrogel with a slow-release function, and a preparation method and use thereof are provided. The polymer hydrogel includes the following components in mass percentage contents: 0.01% to 15% of an active ingredient, 0.01% to 15% of an ion inhibitor, 0.01% to 1% of a crosslinking agent, 0.1% to 10% of a polymer resin, 10% to 35% of a solvent, 0.1% to 15% of a skin-touch regulator, 10% to 55% of deionized water, 0.1% to 3% of an appearance modifier, 0.01% to 1% of a crosslinking regulator, 0.01% to 1% of a preservative, and 0.01% to 5% of a transdermal absorption enhancer. The polymer hydrogel prepared by the present disclosure has excellent skin adaptability and skin permeability, long drug slow-release time, and excellent bioadhesion to the skin, and can be repeatedly peeled off without residue.

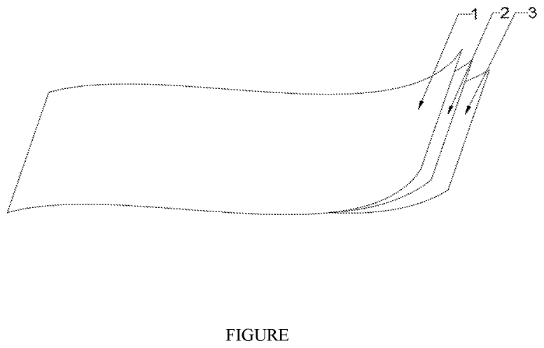

### CROSS REFERENCE TO THE RELATED APPLICATIONS

This application is the national phase entry of International Application No. PCT/CN2022/080351, filed on Mar. 11, 2022, which is based upon and claims priority to Chinese Patent Application No. 202110271604.X, filed on Mar. 12, 2021, and No. 202 110269382.8, filed on Mar. 12, 2021, the entire contents of which are incorporated herein by reference.

### TECHNICAL FIELD

The present disclosure relates to the technical fields of medicines and medical devices and specifically relates to a polymer hydrogel with a slow-release function and a preparation method and use thereof.

### BACKGROUND

With the increasing focus on health, people exercise regularly and inevitably undergo muscle strain. It is necessary to protect muscles before exercise. However, the traditional patch products on the market are easy to fall off from sweating during exercise or need to be used in combination with protective gear, resulting in a bulky use experience.

At present, the development of bandages mainly focuses on the improvement of substrates, for example, a self-adhesive tape, an elastic substrate, or the like may be adopted as a substrate. However, after a skin wound is treated, a bandage directly contacts the skin wound. There is usually spillage near the wound and the bandage usually has poor air permeability, such that it is prone to bacterial infection which causes great pain to a wounded individual and is not conducive to the healing of the wound. The existing bandages with hot melt adhesive coatings cannot play a role of substantial transdermal administration and have a salt-resistant colloidal layout, or some hydrogel bandage products have complex structures designed to overcome the permeability of hydrogels, resulting in the user experience of bulkiness, poor skin adaptation, heavyweight, large thickness, and easy falling-off due to sweating during exercise.

### SUMMARY

In view of the shortcomings in the prior art, an objective of the present disclosure is to provide a polymer hydrogel with a slow-release function and a preparation method and use thereof.

The objective of the present disclosure is achieved by the following technical solutions: The present disclosure provides a polymer hydrogel with a slow-release function, including the following components in mass percentage contents:
to 15% of an active pharmaceutical ingredient, 0.01% to 15% of an ion inhibitor, 0.01% to 1% of a crosslinking agent, 0.1% to 10% of a polymer resin, 10% to 35% of a solvent, 0.1% to 15% of a skin-touch regulator, 10% to 55% of deionized water, 0.1% to 3% of an appearance modifier, 0.01% to 1% of a crosslinking regulator, 0.01% to 1% of a preservative, and 0.01% to 5% of a transdermal absorption enhancer.

Preferably, the polymer hydrogel includes the following components in mass percentage contents:
1% to 10% of an active ingredient, 3% to 10% of an ion inhibitor, 0.1% to 0.5% of a crosslinking agent, 5% to 10% of a polymer resin, 20% to 35% of a solvent, 1% to 5% of a skin-touch regulator, 30% to 55% of deionized water, 0.1% to 1% of an appearance modifier, 0.1% to 1% of a crosslinking regulator, 0.1% to 0.5% of a preservative, and 1% to 5% of a transdermal absorption enhancer.

More preferably, the polymer hydrogel includes the following components in mass percentage contents:
4% to 6% of an active ingredient, 5% of an ion inhibitor, 0.1% to 0.2% of a crosslinking agent, 8% to 9.5% of a polymer resin, 30% to 33% of a solvent, 0.1% of a skin-touch regulator, 40.9% to 52.04% of deionized water, 0.05% to 0.1% of an appearance modifier, 0.2% of a crosslinking regulator, 0.1% of a preservative, and 0.5% of a transdermal absorption enhancer.

Preferably, the active ingredient includes any one or more selected from the group consisting of a pharmaceutical ingredient, a traditional Chinese medicine (TCM) powder or extract, an amino acid, and a plant extract. For example, the pharmaceutical ingredient may be selected from the group consisting of analgesic ingredients such as glucosamine, a capsicum extract, camphor, menthol, methyl salicylate, lidocaine, and lidocaine hydrochloride, and may also be selected from the group consisting of anti-inflammatory and analgesic ingredients such as aspirin, metamizole sodium, acetaminophen, indomethacin, piroxicam, ketorolac, cortisone, hydrocortisone, dexamethasone, glycyrrhetinic acid, biphenylacetic acid, and loxoprofen; the TCM powder or extract may be selected from the group consisting of TCM powders or extracts such as borneol, Aloe Vera, Camellia japonica, a mastic, an Arnica montana flower, Angelica sinensis, a Cassia tora seed, Calendula officinalis, Echinacea purpurea, and a Juniper berry; the amino acid may be selected from the group consisting of glycine, serine, L-tryptophan, arginine, ornithine, 5-hydroxytryptophan, and L-theanine; and the plant extract may be selected from the group consisting of sleep-aiding ingredient-containing plant extracts such as a valerian extract, a passionflower extract, an Anthemis tinctoria extract, a lavender extract, a chamomile extract, a lemon balm extract, a tart cherry extract, a garlic extract, and a spearmint extract, and may also be selected from the group consisting of refreshing plant extracts such as a wintergreen oil, an Angelica dahurica (A. dahurica) extract, an Angelica oil, a cinnamon oil, an Eucalyptus oil, a peppermint oil, a borneol oil, and a Patchouli oil.

Preferably, the ion inhibitor includes at least one selected from the group consisting of nonionic ion inhibitors of polyvinylpyrrolidone (PVP) and polyvinyl alcohol (PVA).

Preferably, the crosslinking agent is aluminum glycinate or aluminum hydroxide.

Preferably, the polymer resin is at least one selected from the group consisting of polyacrylic acid (PAA) and sodium polyacrylate (NaPA).

Preferably, the solvent includes at least one selected from the group consisting of glycerol, propylene glycol (PG), mineral oil, and Polyoxyethylenesorbitan monooleate.

Preferably, the skin-touch regulator is at least one selected from the group consisting of kaolin and sodium carboxymethyl cellulose (CMC-Na); and the appearance modifier is titanium dioxide.

More preferably, the skin-touch regulator is kaolin, which is an oil-absorbing inorganic powder and can regulate oil components secreted on the skin during exercise.

Preferably, the crosslinking regulator is at least one selected from the group consisting of tartaric acid, citric acid, ethylenediaminetetraacetic acid di sodium (EDTA-2Na), ethylenediaminetetraacetic acid tetrasodium (EDTA-4Na), malic acid, and lactic acid; the preservative is at least one selected from the group consisting of benzalkonium chloride, methylparaben, propylparaben, and phenoxyethanol; and the transdermal absorption enhancer is at least one selected from the group consisting of isopropyl myristate, dimethylsulfoxide (DMSO), and azone.

The present disclosure also provides a preparation method of a polymer hydrogel with a slow-release function, including the following steps:
S1. mixing NaPA, the crosslinking agent, the crosslinking regulator, and the appearance modifier with a part of the solvent and stirring a resulting mixture at room temperature for 8 minutes to 15 minutes to achieve thorough dispersion to obtain a phase A;
S2. adding the active pharmaceutical ingredient to the remaining part of the solvent and allowing thorough dissolution at room temperature to obtain a phase B;
S3. mixing the ion inhibitor, the skin-touch regulator, the transdermal absorption enhancer, and the preservative, and stirring a resulting mixture at room temperature for 15 minutes to 20 minutes to obtain a mixed solution, which is a phase C; and
S4. subjecting the phase B and the phase C to emulsification in a homoemulsification machine, pouring an emulsified liquid into a vacuum-stirred reactor, adding the phase A to the vacuum-stirred reactor, and stirring to obtain the polymer hydrogel with a slow-release function.

Preferably, in S4, the emulsification is conducted at a rotational speed of 4,000 r/min for 10 minutes to 15 minutes; and the stirring is conducted at a rotational speed of 40 r/min to 60 r/min for 10 minutes to 15 minutes.

The present disclosure also provides a hydrogel elastic patch, including an elastic material layer, a polymer hydrogel layer with a slow-release function, and a release overlay layer that are arranged sequentially, where the elastic material layer is selected from the group consisting of an elastic material layer compounded with a hydrophobic additive, a material layer formed by compounding an elastic material and a waterproof material, and an elastic material layer with an air layer structure; and the polymer hydrogel layer with a slow-release function includes the polymer hydrogel described above.

Preferably, the release overlay layer is an overlay layer made from any material with a release effect, and is specifically selected from the group consisting of a silicone paper, a pearlescent film, a polypropylene (PP) release film, and a polyethylene terephthalate (PET) release film.

Preferably, the hydrophobic additive is at least one selected from the group consisting of a polyfluoroalkyl acrylate copolymer, a silicone, a fluorocarbon polymer, a long-chain alkane ester, and a copolymer thereof.

Preferably, the waterproof material is a thermoplastic elastomer or a rubber.

Preferably, a preparation method of the elastic material layer compounded with the hydrophobic additive includes: mixing the hydrophobic additive and water in a ratio of 6:94 to 12:88 to obtain a mixed solution; dyeing an elastic material, fixing a color, and finishing; and soaking the elastic material in the mixed solution for 5 minutes, pre-baking the elastic material at 120° C. to 140° C. for 2 minutes, and baking the elastic material at 150° C. to 155° C. for 1 minute, at 160° C. to 165° C. for 2 min, and at 180° C. to 190° C. for 5 minutes to obtain the elastic material layer compounded with the hydrophobic additive.

The present disclosure also provides a preparation method of the hydrogel elastic patch, including the following steps: coating the polymer hydrogel layer with a slow-release function on the elastic material layer, covering the polymer hydrogel layer with the release overlay layer, cutting, curing, and packaging.

Compared with the prior art, the present disclosure has the following beneficial effects:
1. The hydrogel of the present disclosure is prepared as follows: NaPA is introduced into a hydrophilic matrix through crosslinking to obtain a polymer skeleton embedded with an active ingredient, and a non-ionic waterborne ion inhibitor is used for colloidal compounding to improve the salt tolerance of the hydrogel. The hydrophilic polymer hydrogel has excellent skin adaptability and skin permeability, long drug slow-release time, and excellent bioadhesion to the skin, and can be repeatedly peeled off without residue. In particular, when the polymer hydrogel is used by a person during exercise, the polymer hydrogel still retains excellent adhesion even if the person sweats.
2. The hydrogel elastic patch prepared by the present disclosure is suitable for use during exercise and is not easy to fall off. In addition, the elastic material used in the present disclosure has excellent air permeability and prominent elastic deformation capacity, is especially suitable for elbow joints, and enables well adhesion.
3. The existing elastic cloth is very stretchable and has large voids due to high elasticity and the hydrogel is easy to flow out through the voids of the elastic cloth due to its fluidity when coated on the elastic cloth. In the present disclosure, the elastic material layer is subjected to a hydrophobic treatment, or the elastic material is compounded with thermoplastic polyurethane (TPU), which avoids the risk that a hydrogel easily penetrates through an elastic cloth when binding to the elastic cloth, and retains the excellent air permeability of the elastic cloth.

### BRIEF DESCRIPTION OF THE DRAWINGS

Other features, objectives, and advantages of the present disclosure will become more apparent by reading the detailed description of non-limiting embodiments with reference to the following accompanying drawings.

The FIGURE is a structural diagram of the hydrogel elastic patch, where 1 represents a release overlay layer, 2 represents a polymer hydrogel layer with a slow-release function, and 3 represents an elastic material layer.

### DETAILED DESCRIPTION OF THE EMBODIMENTS

The present disclosure is described in detail below with reference to specific examples. The following examples will help those skilled in the art to further understand the present disclosure, but do not limit the present disclosure in any way. It should be noted that those of ordinary skill in the art can further make several variations and improvements without departing from the idea of the present disclosure. These all fall within the protection scope of the present disclosure.

### Example 1

In this example, a polymer hydrogel with a slow-release function was provided, including the following components in mass percentage contents: 4% of lidocaine (active ingredient), 5% of PVP (ion inhibitor), 0.1% of aluminum glycinate (crosslinking agent), 5% of NaPA (polymer resin), 3% of PAA (polymer resin), 24% of glycerol (solvent), 5% of PG (solvent), 1% of Polyoxyethylenesorbitan monooleate (solvent), 0.1% of kaolin (skin-touch regulator), 0.05% of titanium dioxide (appearance modifier), 0.1% of tartaric acid (crosslinking regulator), 0.1% of EDTA-2Na (crosslinking regulator), 0.1% of phenoxyethanol (preservative), 0.5% of DMSO (transdermal absorption enhancer), and 51.95% of deionized water.

A preparation method of the polymer hydrogel was as follows:
(1) NaPA, aluminum glycinate, kaolin, EDTA-2Na, tartaric acid, and glycerol were mixed and stirred at room temperature for 10 minutes to obtain a phase A.
(2) Lidocaine was mixed with PG and Polyoxyethylenesorbitan monooleate, and a resulting mixture was subjected to thorough dissolution at room temperature to obtain a phase B.
(3) PVP, titanium dioxide, DMSO, and phenoxyethanol were added to deionized water and a resulting mixture was stirred at room temperature for 15 minutes to 20 minutes to obtain a mixed solution, which was a phase C.
(4) The phase B and the phase C were subjected to emulsification for 10 minutes to 15 minutes at a rotational speed of 4,000 r/min in a homoemulsification machine, an emulsified liquid was poured into a vacuum-stirred reactor, then the phase A was added to the vacuum-stirred reactor, and a resulting mixture was stirred for 10 minutes to 15 minutes at a rotational speed of 40 r/min to 60 r/min to obtain a hydrogel paste with the active pharmaceutical ingredient embedded.

### Example 2

In this example, a polymer hydrogel with a slow-release function was provided, including the following components in mass percentage contents: 5% of menthol (active ingredient), 5% of PVP (ion inhibitor), 0.1% of aluminum glycinate (crosslinking agent), 5.5% of NaPA (polymer), 3.5% of PAA (polymer), 23% of glycerol (solvent), 7% of PG (solvent), 1% of Polyoxyethylenesorbitan monooleate (solvent), 0.1% of CMC-Na (skin-touch regulator), 0.1% of titanium dioxide (appearance modifier), 0.1% of tartaric acid (crosslinking regulator), 0.1% of EDTA-2Na (crosslinking regulator), 0.1% of benzalkonium chloride (preservative), 0.5% of isopropyl myristate (transdermal absorption enhancer), and 48.9% of deionized water.

A preparation method of the polymer hydrogel was as follows:
(1) NaPA, aluminum glycinate, CMC-Na, EDTA-2Na, tartaric acid, and glycerol were mixed and stirred at room temperature for 10 minutes to obtain a phase A.
(2) Menthol was mixed with PG and Polyoxyethylenesorbitan monooleate and a resulting mixture was subjected to thorough dissolution at room temperature to obtain a phase B.
(3) PVP, titanium dioxide, isopropyl myristate, and phenoxyethanol were added to deionized water and a resulting mixture was stirred at room temperature for 15 minutes to 20 minutes to obtain a mixed solution, which was a phase C.
(4) The phase B and the phase C were subjected to emulsification for 10 minutes to 15 minutes at a rotational speed of 4,000 r/min in a homoemulsification machine, an emulsified liquid was poured into a vacuum-stirred reactor, then the phase A was added to the vacuum-stirred reactor, and a resulting mixture was stirred for 10 minutes to 15 minutes at a rotational speed of 40 r/min to 60 r/min to obtain a hydrogel paste with the active ingredient embedded.

### Example 3

In this example, a polymer hydrogel with a slow-release function was provided, including the following components in mass percentage contents: 5% of menthol (active ingredient), 5% of PVP (ion inhibitor), 0.2% of aluminum hydroxide (crosslinking agent), 3.5% of NaPA (polymer), 6% of PAA (polymer), 25% of glycerol (solvent), 7% of PG (solvent), 1% of Polyoxyethylenesorbitan monooleate (solvent), 0.1% of CMC-Na (skin-touch regulator), 0.1% of titanium dioxide (appearance modifier), 0.1% of tartaric acid (crosslinking regulator), 0.1% of EDTA-2Na (crosslinking regulator), 0.1% of methylparaben (preservative), 0.1% of propylparaben (preservative), 0.5% of isopropyl myristate (transdermal absorption enhancer), and 46.2% of deionized water.

A preparation method of the polymer hydrogel was as follows:
(1) NaPA, aluminum hydroxide, CMC-Na, EDTA-2Na, tartaric acid, and glycerol were mixed and stirred at room temperature for 10 minutes to obtain a phase A.
(2) Menthol was mixed with PG and Polyoxyethylenesorbitan monooleate and a resulting mixture was subjected to thorough dissolution at room temperature to obtain a phase B.
(3) PVP, titanium dioxide, isopropyl myristate, methylparaben, and propylparaben were added to deionized water and a resulting mixture was stirred at room temperature for 15 minutes to 20 minutes to obtain a mixed solution, which was a phase C.
(4) The phase B and the phase C were subjected to emulsification for 10 minutes to 15 minutes at a rotational speed of 4,000 r/min in a homoemulsification machine, an emulsified liquid was poured into a vacuum-stirred reactor, then the phase A was added to the vacuum-stirred reactor, and a resulting mixture was stirred for 10 minutes to 15 minutes at a rotational speed of 40 r/min to 60 r/min to obtain a hydrogel paste with the active ingredient embedded.

### Example 4

In this example, a polymer hydrogel with a slow-release function was provided, and a composition of the polymer hydrogel was basically the same as that of Example 1, except that: PVA was used instead of PVP in this example.

The hydrogels prepared in Examples 1 to 4 have excellent skin adaptability and skin permeability, long drug slow-release time, and excellent bioadhesion to the skin and can be repeatedly peeled off without residue. In particular, when the hydrogels are used by a person during exercise, the hydrogels still retain excellent adhesion even if the person sweats.

### Example 5

In this example, a polymer hydrogel with a slow-release function was provided and a composition of the polymer hydrogel was basically the same as that of Example 1, except that: PVA was used instead of PVP, a content of PVA was 3%, and a content of deionized water was 53.95% in this example.

### Example 6

In this example, a polymer hydrogel with a slow-release function was provided and a composition of the polymer hydrogel was basically the same as that of Example 1, except that: PVA was used instead of PVP, a content of PVA was 8%, and a content of deionized water was 48.95% in this example.

### Example 7

In this example, a polymer hydrogel with a slow-release function was provided and a composition of the polymer hydrogel was basically the same as that of Example 1, except that: PVA was used instead of PVP, a content of PVA was 10%, and a content of deionized water was 46.95% in this example.

### Comparative Example 1

In this comparative example, a polymer hydrogel with a slow-release function was provided, and a composition of the polymer hydrogel was basically the same as that of Example 1, except that: the crosslinking agent was not added and a content of deionized water was 52.14% in this comparative example.

A preparation method of the polymer hydrogel was the same as that of Example 1.

Because the crosslinking agent was not added, a crosslinking reaction occurred too fast or too slowly, and a crosslinking degree of the hydrogel was uneven, resulting in failed coating.

### Comparative Example 2

In this comparative example, a polymer hydrogel with a slow-release function was provided, and a composition of the polymer hydrogel was basically the same as that of Example 1, except that: the ion inhibitor was not added and a content of deionized water was 56.95% in this comparative example.

A preparation method of the polymer hydrogel was the same as that of Example 1.

Effectiveness Verification:

The hydrogel pastes prepared in Example 1 and Comparative Example 2 each were coated on an elastic cloth and then covered with a release overlay layer, and a resulting product was cut and cured to obtain a hydrogel paste patch. The prepared hydrogel paste patches each were tested for efficacy, and a specific test method and test results were as follows:

The hydrogel paste patches of Example 1 and Comparative Example 2 each were cut into three 30 cm*2.5 cm strip samples. About 2 g of an artificial sweat was evenly applied to a surface of each hydrogel paste patch, a pre-treatment was conducted at 25±2° C. and 60% RH for 2 h, and a peeling strength was tested according to GB/T 2792-2014. Test results were shown in Table 1 below:

TABLE 1

| | | | | Sample of Comparative Example 2 | | |
|---|---|---|---|---|---|---|
| | Sample of Example 1 | | | Test | Test | Test |
| | Sample 1 | Sample 2 | Sample 3 | sample 1 | sample 2 | sample 3 |
| Peeling strength | 1.012 | 1.134 | 1.109 | 0.235 | 0.206 | 0.307 |
| (N/2.5 cm) | | | | | | |

It can be seen from the above test results that, since the ion inhibitor is not added in Comparative Example 2, the adhesion effect is significantly reduced after the immersion of the artificial sweat.

The hydrogel paste patches of Examples 2 to 7 each were cut into three 30 cm*2.5 cm strip samples according to the above method. About 2 g of an artificial sweat was evenly applied to a surface of each hydrogel paste patch, a pre-treatment was conducted at 25±2° C. and 60% RH for 2 h, and a peeling strength was tested according to GB/T 2792-2014. Test results (an average of the three samples) were shown in Table 2 below:

TABLE 2

| | Sample of | Sample of | Sample of | Sample of | Sample of | Sample of |
|---|---|---|---|---|---|---|
| | Example 2 | Example 3 | Example 4 | Example 5 | Example 6 | Example 7 |
| Peeling strength | 1.124 | 1.036 | 1.305 | 0.523 | 0.632 | 0.276 |
| (N/2.5 cm) | | | | | | |

### Example 8

In this example, a hydrogel elastic patch was provided, as shown in the FIGURE, including a release overlay layer 1, a polymer hydrogel layer 2 with a slow-release function, and an elastic material layer 3 that were arranged sequentially. The elastic material layer 3 was an elastic cloth compounded with a hydrophobic additive, and the release overlay layer 1 was a pearlescent film.

The polymer hydrogel layer with a slow-release function included the following components in mass percentage contents: 4% of lidocaine (active pharmaceutical ingredient), 5% of PVP (ion inhibitor), 0.1% of aluminum glycinate (crosslinking agent), 5% of NaPA (polymer resin), 3% of PAA (polymer resin), 24% of glycerol (solvent), 5% of PG (solvent), 1% of Polyoxyethylenesorbitan monooleate (solvent), 0.1% of kaolin (skin-touch regulator), 0.05% of titanium dioxide (appearance modifier), 0.1% of tartaric acid (crosslinking regulator), 0.1% of EDTA-2Na (crosslinking regulator), 0.1% of phenoxyethanol (preservative), 0.5% of DMSO (transdermal absorption enhancer), and 51.95% of deionized water.

A preparation method of the elastic cloth compounded with the hydrophobic additive was as follows: a polyfluoroalkyl acrylate copolymer (hydrophobic additive) and water were mixed in a ratio of 6:94 to obtain a mixed solution; an elastic material was dyed, subjected to color fixation, and finished; and then the elastic material was soaked in the mixed solution for 5 minutes, pre-baked at 120° C. to 140° C. for 2 minutes, and baked at 150° C. to 155° C. for 1 minute, at 160° C. to 165° C. for 2 minutes, and at 180° C. to 190° C. for 5 minutes. According to the test method in AATCC-127-1977, a hydrophobic level of the elastic cloth could reach 5 or more.

A preparation method of the hydrogel elastic patch in this example was as follows:
(1) NaPA, aluminum glycinate, kaolin, EDTA-2Na, tartaric acid, and glycerol were mixed and stirred at room temperature for 10 minutes to obtain a phase A.
(2) Lidocaine was mixed with PG and Polyoxyethylenesorbitan monooleate, and a resulting mixture was subjected to thorough dissolution at room temperature to obtain a phase B.
(3) PVP, titanium dioxide, DMSO, and phenoxyethanol were added to deionized water, and a resulting mixture was stirred at room temperature for 15 minutes to 20 minutes to obtain a mixed solution, which was a phase C.
(4) The phase B and the phase C were subjected to emulsification for 10 minutes to 15 minutes at a rotational speed of 4,000 r/min in a homoemulsification machine, an emulsified liquid was poured into a vacuum-stirred reactor, then the phase A was added to the vacuum-stirred reactor, and a resulting mixture was stirred for 10 minutes to 15 minutes at a rotational speed of 40 r/min to 60 r/min to obtain a polymer hydrogel with a slow-release function.
(5) The polymer hydrogel with a slow-release function was coated on the elastic cloth compounded with the hydrophobic additive, and then covered with a pearlescent film, and a resulting product was cut, cured, and packaged.

In the hydrogel elastic patch prepared in this example, the hydrogel did not penetrate the elastic cloth due to the hydrophobic treatment of the elastic cloth.

### Example 9

In this example, a hydrogel elastic patch was provided, as shown in the FIGURE, including a release overlay layer 1, a polymer hydrogel layer 2 with a slow-release function, and an elastic material layer 3 that were arranged sequentially. The elastic material layer 3 was an elastic cloth compounded with a hydrophobic additive, and the release overlay layer 1 was a pearlescent film.

The polymer hydrogel layer with a slow-release function included the following components in mass percentage contents: 5% of menthol (active ingredient), 5% of PVP (ion inhibitor), 0.1% of aluminum glycinate (crosslinking agent), 5.5% of NaPA (polymer), 3.5% of PAA (polymer), 23% of glycerol (solvent), 7% of PG (solvent), 1% of Polyoxyethylenesorbitan monooleate (solvent), 0.1% of CMC-Na (skin-touch regulator), 0.1% of titanium dioxide (appearance modifier), 0.1% of tartaric acid (crosslinking regulator), 0.1% of EDTA-2Na (crosslinking regulator), 0.1% of benzalkonium chloride (preservative), 0.5% of isopropyl myristate (transdermal absorption enhancer), and 48.9% of deionized water.

A preparation method of the elastic cloth compounded with the hydrophobic additive was as follows: a polyfluoroalkyl acrylate copolymer (hydrophobic additive) and water were mixed in a ratio of 8:92 to obtain a mixed solution; an elastic material was dyed, subjected to color fixation, and finished; and then the elastic material was soaked in the mixed solution for 5 minutes, pre-baked at 120° C. to 140° C. for 2 minutes, and baked at 150° C. to 155° C. for 1 minute, at 160° C. to 165° C. for 2 minutes, and at 180° C. to 190° C. for 5 minutes. According to the test method in AATCC-127-1977, a hydrophobic level of the elastic cloth could reach 5 or more.

A preparation method of the hydrogel elastic patch in this example was as follows:
(1) NaPA, aluminum glycinate, CMC-Na, EDTA-2Na, tartaric acid, and glycerol were mixed and stirred at room temperature for 10 min to obtain a phase A.
(2) Menthol was mixed with PG and Polyoxyethylenesorbitan monooleate and a resulting mixture was subjected to thorough dissolution at room temperature to obtain a phase B.
(3) PVP, titanium dioxide, isopropyl myristate, and phenoxyethanol were added to deionized water, and a resulting mixture was stirred at room temperature for 15 minutes to 20 minutes to obtain a mixed solution, which was a phase C.
(4) The phase B and the phase C were subjected to emulsification for 10 minutes to 15 minutes at a rotational speed of 4,000 r/min in a homoemulsification machine, an emulsified liquid was poured into a vacuum-stirred reactor, then the phase A was added to the vacuum-stirred reactor, and a resulting mixture was stirred for 10 minutes to 15 minutes at a rotational speed of 40 r/min to 60 r/min to obtain a hydrogel paste with the active ingredient embedded.
(5) The polymer hydrogel with a slow-release function was coated on the elastic cloth compounded with the hydrophobic additive, and then covered with a pearlescent film, and a resulting product was cut, cured, and packaged.

In the hydrogel elastic patch prepared in this example, the hydrogel did not penetrate the elastic cloth due to the hydrophobic treatment of the elastic cloth.

### Example 10

In this example, a hydrogel elastic patch was provided, as shown in the FIGURE, including a release overlay layer 1, a polymer hydrogel layer 2 with a slow-release function, and an elastic material layer 3 that were arranged sequentially. The elastic material layer 3 was an elastic cloth compounded with a hydrophobic additive, and the release overlay layer 1 was a PP release film.

The polymer hydrogel layer with a slow-release function included the following components in mass percentage contents: 5% of menthol (active pharmaceutical ingredient), 5% of PVP (ion inhibitor), 0.2% of aluminum hydroxide (crosslinking agent), 3.5% of NaPA (polymer), 6% of PAA (polymer), 25% of glycerol (solvent), 7% of PG (solvent), 1% of Polyoxyethylenesorbitan monooleate (solvent), 0.1% of CMC-Na (skin-touch regulator), 0.1% of titanium dioxide (appearance modifier), 0.1% of tartaric acid (crosslinking regulator), 0.1% of EDTA-2Na (crosslinking regulator), 0.1% of methylparaben (preservative), 0.1% of propylparaben (preservative), 0.5% of isopropyl myristate (transdermal absorption enhancer), and 46.2% of deionized water.

A preparation method of the elastic cloth compounded with the hydrophobic additive was as follows: a polyfluoroalkyl acrylate copolymer (hydrophobic additive) and water were mixed in a ratio of 9:91 to obtain a mixed solution; an elastic material was dyed, subjected to color fixation, and finished; and then the elastic material was soaked in the mixed solution for 5 minutes, pre-baked at 120° C. to 140° C. for 2 minutes, and baked at 150° C. to 155° C. for 1 minute, at 160° C. to 165° C. for 2 minutes, and at 180° C. to 190° C. for 5 minutes. According to the test method in AATCC-127-1977, a hydrophobic level of the elastic cloth could reach 5 or more.

A preparation method of the hydrogel elastic patch was as follows:
(1) NaPA, aluminum hydroxide, CMC-Na, EDTA-2Na, tartaric acid, and glycerol were mixed and stirred at room temperature for 10 minutes to obtain a phase A.
(2) Menthol was mixed with PG and Polyoxyethylenesorbitan monooleate and a resulting mixture was subjected to thorough dissolution at room temperature to obtain a phase B.
(3) PVP, titanium dioxide, isopropyl myristate, methylparaben, and propylparaben were added to deionized water, and a resulting mixture was stirred at room temperature for 15 minutes to 20 minutes to obtain a mixed solution, which was a phase C.
(4) The phase B and the phase C were subjected to emulsification for 10 minutes to 15 minutes at a rotational speed of 4,000 r/min in a homoemulsification machine, an emulsified liquid was poured into a vacuum-stirred reactor, then the phase A was added to the vacuum-stirred reactor, and a resulting mixture was stirred for 10 minutes to 15 minutes at a rotational speed of 40 r/min to 60 r/min to obtain a hydrogel paste with the active ingredient embedded.
(5) The polymer hydrogel with a slow-release function was coated on the elastic cloth compounded with the hydrophobic additive, and then covered with a PP release film, and a resulting product was cut, cured, and packaged.

In the hydrogel elastic patch prepared in this example, the hydrogel did not penetrate the elastic cloth due to the hydrophobic treatment of the elastic cloth.

### Example 11

In this example, a hydrogel elastic patch was provided, and a structure of the hydrogel elastic patch was basically the same as that of Example 8, except that: PVA was used instead of PVP in the composition of the polymer hydrogel with a slow-release function in this example.

The hydrogels prepared in Examples 8 to 11 have excellent skin adaptability and skin permeability, long drug slow-release time, and excellent bioadhesion to the skin, and can be repeatedly peeled off without residue. In particular, when the hydrogels are used by a person during exercise, the hydrogels still retain excellent adhesion even if the person sweats.

### Example 12

In this example, a hydrogel elastic patch was provided, and a structure of the hydrogel elastic patch was basically the same as that of Example 8, except that: PVA was used instead of PVP in the composition of the polymer hydrogel with a slow-release function, a content of PVA was 3%, and a content of deionized water was 53.95% in this example.

### Example 13

In this example, a hydrogel elastic patch was provided, and a structure of the hydrogel elastic patch was basically the same as that of Example 8, except that: PVA was used instead of PVP in the composition of the polymer hydrogel with a slow-release function, a content of PVA was 8%, and a content of deionized water was 48.95% in this example.

### Example 14

In this example, a hydrogel elastic patch was provided, and a structure of the hydrogel elastic patch was basically the same as that of Example 8, except that: PVA was used instead of PVP in the composition of the polymer hydrogel with a slow-release function, a content of PVA was 10%, and a content of deionized water was 46.95% in this example.

### Comparative Example 3

In this comparative example, a hydrogel elastic patch was provided, and a structural composition of the hydrogel elastic patch was basically the same as that of Example 8, except that: the crosslinking agent was not added in the polymer hydrogel layer with a slow-release function and a content of deionized water was 52.14% in this comparative example.

A preparation method of the polymer hydrogel was the same as that of Example 8.

Because the crosslinking agent was not added in the polymer hydrogel layer of the hydrogel elastic patch prepared in this comparative example, a crosslinking reaction occurred too fast or too slowly, and a crosslinking degree of the hydrogel was uneven, resulting in failed coating.

### Comparative Example 4

In this comparative example, a hydrogel elastic patch was provided, and a structural composition of the hydrogel elastic patch was basically the same as that of Example 8, except that: the ion inhibitor was not added in the polymer hydrogel layer with a slow-release function and a content of deionized water was 56.95% in this comparative example.

A preparation method of the polymer hydrogel was the same as that of Example 8.

After being applied for a long time, the hydrogel elastic patch prepared in this comparative example was easy to fall off due to sweating.

### Comparative Example 5

In this comparative example, a hydrogel elastic patch was provided, and a structural composition of the hydrogel elastic patch was basically the same as that of Example 8, except that: the elastic material layer used in this comparative example was a conventional elastic cloth (namely, an elastic cloth without a hydrophobic additive).

In the hydrogel elastic patch prepared in this comparative example, the hydrogel penetrated the elastic cloth because the elastic cloth was not subjected to a hydrophobic treatment.

Effectiveness Verification:

The hydrogel elastic patches prepared in Example 8 and Comparative Example 4 each were cut into three 30 cm*2.5 cm strip samples. About 2 g of an artificial sweat was evenly applied to a surface of each hydrogel elastic patch, a pre-treatment was conducted at 25±2° C. and 60% RH for 2 h, and a peeling strength was tested according to GB/T 2792-2014. Test results were shown in Table 3 below:

TABLE 3

| | | | | Sample of Comparative Example 4 | | |
|---|---|---|---|---|---|---|
| | Sample of Example 8 | | | Test | Test | Test |
| | Sample 1 | Sample 2 | Sample 3 | sample 1 | sample 2 | sample 3 |
| Peeling strength | 1.012 | 1.134 | 1.109 | 0.235 | 0.206 | 0.307 |
| (N/25 cm) | | | | | | |

It can be seen from the above test results that, since the ion inhibitor is not added in Comparative Example 4, the adhesion effect is significantly reduced after the immersion of the artificial sweat.

The hydrogel elastic patches prepared in Examples 9 to 14 each were cut into three 30 cm*2.5 cm strip samples according to the above method. About 2 g of an artificial sweat was evenly applied to a surface of each hydrogel elastic patch, a pre-treatment was conducted at 25±2° C. and 60% RH for 2 h, and a peeling strength was tested according to GB/T 2792-2014. Test results (an average of the three samples) were shown in Table 4 below:

TABLE 4

| | Sample of | Sample of | Sample of | Sample of | Sample of | Sample of |
|---|---|---|---|---|---|---|
| | Example 9 | Example 10 | Example 11 | Example 12 | Example 13 | Example 14 |
| Peeling strength | 1.124 | 1.036 | 1.305 | 0.523 | 0.632 | 0.276 |
| (N/25 cm) | | | | | | |

In the present disclosure, the hydrogel may also be coated on a waterproof composite material or an air layer material with a specified structure to solve the problem of hydrogel exudation; and a shape of the product is not limited to those illustrated in the accompanying drawings, and the product can be cut into any size; and the product with a hollowed surface exhibits improved air permeability.

The examples are described above to facilitate the comprehension and use of the present disclosure by those of ordinary skill in the art. Obviously, those skilled in the art can easily make various modifications to these examples, and apply a general principle described herein to other examples without creative efforts. Therefore, the present disclosure is not limited to the above examples. All improvements and modifications made by a person skilled in the art according to the disclosure of the present disclosure should fall within the protection scope of the present disclosure.

What is claimed is:

1. A polymer hydrogel with a slow-release function, comprising the following components in mass percentage contents:
to 15% of an active ingredient, 0.01% to 15% of an ion inhibitor, 0.01% to 1% of a crosslinking agent, 0.1% to 10% of a polymer resin, 10% to 35% of a solvent, 0.1% to 15% of a skin-touch regulator, 10% to 55% of deionized water, 0.1% to 3% of an appearance modifier, 0.01% to 1% of a crosslinking regulator, 0.01% to 1% of a preservative, and 0.01% to 5% of a transdermal absorption enhancer.

2. The polymer hydrogel with the slow-release function according to claim 1, wherein the active ingredient comprises any one or more selected from the group consisting of a pharmaceutical ingredient, a traditional Chinese medicine (TCM) powder or an extract of the TCM powder, an amino acid, and a plant extract.

3. The polymer hydrogel with the slow-release function according to claim 1, wherein the ion inhibitor comprises at least one selected from the group consisting of nonionic ion inhibitors of polyvinylpyrrolidone (PVP) and polyvinyl alcohol (PVA).

4. The polymer hydrogel with the slow-release function according to claim 1, wherein the crosslinking agent is aluminum glycinate or aluminum hydroxide.

5. The polymer hydrogel with the slow-release function according to claim 1, wherein the polymer resin is at least one selected from the group consisting of polyacrylic acid (PAA) and sodium polyacrylate (NaPA).

6. The polymer hydrogel with the slow-release function according to claim 1, wherein the solvent comprises at least one selected from the group consisting of glycerol, propylene glycol (PG), mineral oil, and Polyoxyethylenesorbitan monooleate.

7. The polymer hydrogel with the slow-release function according to claim 1, wherein the skin-touch regulator is at least one selected from the group consisting of kaolin and sodium carboxymethyl cellulose (CMC-Na); and the appearance modifier is titanium dioxide.

8. The polymer hydrogel with the slow-release function according to claim 1, wherein the crosslinking regulator is at least one selected from the group consisting of tartaric acid, citric acid, ethylenediaminetetraacetic acid disodium (EDTA-2Na), ethylenediaminetetraacetic acid tetrasodium (EDTA-4Na), malic acid, and lactic acid; the preservative is at least one selected from the group consisting of benzalkonium chloride, methylparaben, propylparaben, and phenoxyethanol; and the transdermal absorption enhancer is at least one selected from the group consisting of isopropyl myristate, dimethylsulfoxide (DMSO), and azone.

9. A preparation method of the polymer hydrogel with the slow-release function according to claim 1, comprising the following steps:
S1. mixing NaPA, the crosslinking agent, the crosslinking regulator, and the appearance modifier with a part of the solvent, and stirring a first resulting mixture at room temperature for 8 minutes to 15 minutes to achieve thorough dispersion to obtain a phase A;
S2. adding the active ingredient to a remaining part of the solvent, and allowing thorough dissolution at room temperature to obtain a phase B;
S3. mixing the ion inhibitor, the skin-touch regulator, the transdermal absorption enhancer, and the preservative, and stirring a second resulting mixture at room temperature for 15 minutes to 20 minutes to obtain a mixed solution, which is a phase C; and
S4. subjecting the phase B and the phase C to emulsification in a homoemulsification machine, pouring a resulting emulsified liquid into a vacuum-stirred reactor, adding the phase A to the vacuum-stirred reactor, and stirring to obtain the polymer hydrogel with the slow-release function.

10. The preparation method of the polymer hydrogel with the slow-release function according to claim 9, wherein in S4, the emulsification is conducted at a rotational speed of 4,000 r/min for 10 minutes to 15 minutes; and the stirring is conducted at a rotational speed of 40 r/min to 60 r/min for 10 minutes to 15 minutes.

11. A hydrogel elastic patch, comprising an elastic material layer, a polymer hydrogel layer with the slow-release function, and a release overlay layer, wherein the elastic material layer, the polymer hydrogel layer with the slow-release function, and the release overlay layer are arranged sequentially, the elastic material layer is selected from the group consisting of an elastic material layer compounded with a hydrophobic additive, a material layer formed by compounding an elastic material and a waterproof material, and an elastic material layer with an air layer structure; and the polymer hydrogel layer comprises the polymer hydrogel according to claim 1.

12. The hydrogel elastic patch according to claim 11, wherein the hydrophobic additive is at least one selected from the group consisting of a polyfluoroalkyl acrylate copolymer, a silicone, a fluorocarbon polymer, a long-chain alkane ester, and a copolymer of the long-chain alkane ester; and the waterproof material is a thermoplastic elastomer or a rubber.

13. The hydrogel elastic patch according to claim 11, wherein a preparation method of the elastic material layer compounded with the hydrophobic additive comprises: mixing the hydrophobic additive and water in a ratio of 6:94 to 12:88 to obtain a mixed solution; dyeing an elastic material, fixing a color, and finishing; and soaking the elastic material in the mixed solution for 5 minutes, pre-baking the elastic material at 120° C. to 140° C. for 2 min, and baking the elastic material at 150° C. to 155° C. for 1 minute, at 160° C. to 165° C. for 2 minutes, and at 180° C. to 190° C. for 5 minutes to obtain the elastic material layer compounded with the hydrophobic additive.

14. A preparation method of the hydrogel elastic patch according to claim 11, comprising the following steps: coating the polymer hydrogel layer with the slow-release function on the elastic material layer, covering the polymer hydrogel layer with the release overlay layer, cutting, curing, and packaging to obtain the hydrogel elastic patch.

15. The hydrogel elastic patch according to claim 12, wherein a preparation method of the elastic material layer compounded with the hydrophobic additive comprises: mixing the hydrophobic additive and water in a ratio of 6:94 to 12:88 to obtain a mixed solution; dyeing an elastic material, fixing a color, and finishing; and soaking the elastic material in the mixed solution for 5 minutes, pre-baking the elastic material at 120° C. to 140° C. for 2 min, and baking the elastic material at 150° C. to 155° C. for 1 minute, at 160° C. to 165° C. for 2 minutes, and at 180° C. to 190° C. for 5 minutes to obtain the elastic material layer compounded with the hydrophobic additive.
